(19) United States
(12) Patent Application Publication
(10) Pub. No.: US 2009/0210040 A1
(43) Pub. Date: Aug. 20, 2009

(54) VARIABLE LENGTH MEDICAL ELECTRICAL STIMULATION LEAD

(76) Inventor: Francisco OCHOA, Cudahy, CA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 12/033,244

(22) Filed: Feb. 19, 2008

(51) Int. Cl. A61N 1/05

(52) U.S. Cl. 607/116

(57) ABSTRACT

An apparatus and method for an electrical stimulation lead having a selectively variable length. In an embodiment of the invention, an apparatus includes a conducting element, a stimulating electrode, a pickup electrode and a sheath. The conductive element has a proximal end, a distal end and a length which is defined between the proximal and distal ends. The stimulating electrode is coupled to the distal end of the conductive element and the pickup electrode is coupled to the proximal end of the conductive element. The sheath of the apparatus is configured to enclose at least a portion of the conductive element. The sheath has a reconfigurable portion that is able to move between a first configuration and a second configuration. The sheath has a first length when in the first configuration and a second length when in the second configuration.

### BACKGROUND

This invention relates generally to electrical stimulation leads, and more particularly, to an electrical stimulation lead having a selectively variable length.

Implantable electrical stimulation leads can include a stimulating electrode located adjacent a target nerve location and a pickup electrode adjacent an external generator location. To accommodate for each of the locations and the potentially large variation in human anatomy, electrical leads come in different lengths. For example, the two leads illustrated in FIG. 1 have a substantially common stimulating electrode location but have different pickup electrode locations. Said another way, Lead 1 is necessarily longer than Lead 2 to account for different locations of their associated pulse generators. In FIG. 2, the leads have common stimulation electrode locations as well as pickup electrode locations because the two leads are associated with a single pulse generator and are implanted adjacent the same nerve. Lead 2, however, is longer than Lead 1, resulting in the need to alter Lead 2 in a loop configuration to place the pickup electrode in an appropriate location. Any excess lead length can be problematic for the placement and functionality of the lead. As a result, manufacturers produce a wide assortment of constant length leads. In the long run, this assortment introduces manufacturing, inventory and instrumentation problems. Additionally, it is usually not known what the required lead length is until the late stages of the implantation, which is an additional challenge in using fixed length leads.

What is needed is a variable length lead that accommodates for the variation in human anatomy, surgical techniques, and stimulation configurations.

### SUMMARY

In an embodiment of the invention, an apparatus includes a conducting element, a stimulating electrode, a pickup electrode and a sheath. The conductive element has a proximal end, a distal end and a length which is defined between the proximal and distal ends. The stimulating electrode is coupled to the distal end of the conductive element and the pickup electrode is coupled to the proximal end of the conductive element. The sheath of the apparatus is configured to enclose at least a portion of the conductive element. The sheath has a reconfigurable portion that is able to move between a first configuration and a second configuration. The sheath has a first length when in the first configuration and a second length when in the second configuration.

In another embodiment of the invention, the apparatus includes a conducting element, a stimulating electrode, a pickup electrode and a monolithic sheath. As in the previous embodiment, the conductive element has a proximal end, a distal end and a length that is defined between the proximal and distal ends. Similarly, the stimulating electrode is coupled to the distal end of the conductive element and the pickup electrode is coupled to the proximal end of the conductive element. In some embodiments, the apparatus has a monolithic sheath that is configured to enclose at least a portion of the conductive element. At least a portion of the sheath is reconfigurable to vary its length.

A method according to an embodiment of the invention includes inserting an electrical stimulation lead into the body and varying the length of its sheath by moving a reconfigurable portion of the sheath between a first configuration and a second configuration. The length of the sheath in the first configuration is different from the length of the sheath in the second configuration.

### BRIEF DESCRIPTION OF THE DRAWINGS

FIG. 1 is an illustration of two stimulating leads targeting a similar nerve location but with different pickup electrode locations.

FIG. 2 is an illustration of two stimulating leads targeting the same nerve location where one lead is longer than the other.

FIG. 3 is a schematic illustration of an apparatus according to an embodiment of the invention with a single reconfigurable portion in a first configuration.

FIG. 4 is a schematic illustration of the apparatus illustrated in FIG. 3 in a second configuration.

FIG. 5 is a schematic illustration of an apparatus according to an embodiment of the invention with multiple reconfigurable portions in a first configuration.

FIG. 6 is a schematic illustration of the apparatus illustrated in FIG. 5 in a second configuration.

FIG. 7 is schematic illustration of an apparatus according to an embodiment of the invention in a first configuration.

FIG. 8 is a schematic illustration of the apparatus illustrated in FIG. 7 in a second configuration.

FIG. 9 is a schematic illustration of the apparatus illustrated in FIG. 7 in an alternative second configuration.

FIG. 10 is a schematic illustration of an apparatus according to an embodiment of the invention in a first configuration.

FIG. 11 is a schematic illustration of the apparatus illustrated in FIG. 10 in a second configuration.

FIG. 12 is a schematic illustration of apparatus illustrated in FIG. 10 in an alternative second configuration.

FIG. 13 is a schematic illustration of an apparatus according to an embodiment of the invention showing the conductive element in a first configuration.

FIG. 14 is a schematic illustration of the apparatus illustrated in FIG. 13 showing the conductive element in a second configuration.

FIG. 15 is a plan view of an apparatus according to an embodiment of the invention in a first configuration.

FIG. 16 is a cross-sectional illustration of the apparatus illustrated in FIG. 8.

FIG. 17 is a plan view of the embodiment illustrated in FIG. 8 in a second configuration.

FIG. 18 is a cross-sectional illustration of the apparatus illustrated in FIG. 9.

FIG. 19 is a schematic illustration of an apparatus according to an embodiment of the invention having a corrugated reconfigurable portion.

FIG. 20 is a flow chart of a method according to an embodiment of the invention.

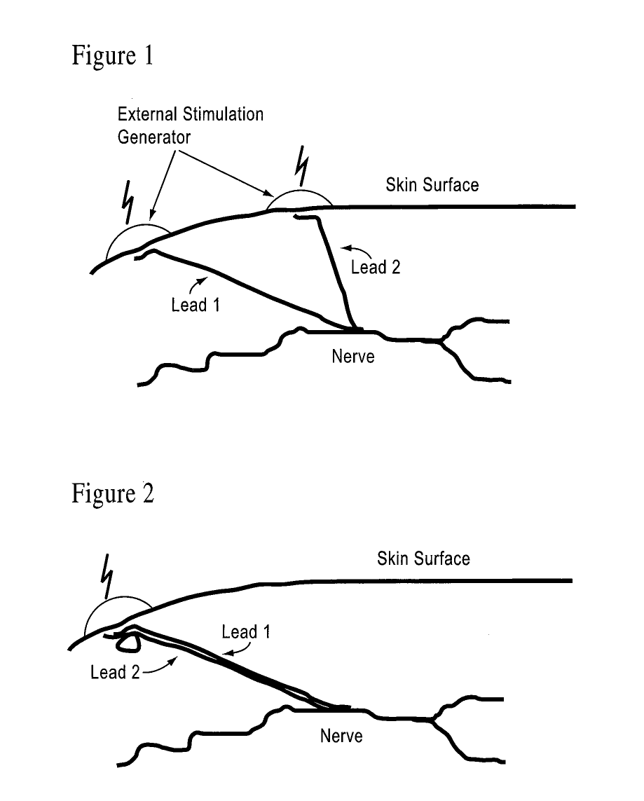

### DETAILED DESCRIPTION

In some embodiments, an apparatus includes an electrical stimulation lead that is configured to be implanted within a body. The electrical lead includes a conductive element, a stimulating electrode, a pickup electrode and a sheath. The pickup electrode is coupled to the proximal end of the conductive element and is configured to receive electrical signals from an external stimulation generator. The stimulating electrode is coupled to the distal end of the conductive element and is configured to stimulate a targeted site within the body. The sheath is configured to enclose at least a portion of the conductive element. The sheath includes a reconfigurable portion that allows the length of the sheath to vary. In some embodiments, the sheath is moveable between a first configuration and a second configuration.

In some embodiments, a kit includes an electrical stimulation lead that is configured to be implanted within a body. The electrical lead includes a conductive element, a stimulating electrode, a pickup electrode and a sheath. The pickup electrode is coupled to the proximal end of the conductive element and is configured to receive electrical signals from an external stimulation generator. The stimulating electrode is coupled to the distal end of the conductive element and is configured to stimulate a targeted site within the body. The sheath is configured to enclose at least a portion of the conductive element. The sheath includes a reconfigurable portion that allows the length of the sheath to vary.

In some embodiments, a method includes inserting an electrical stimulation lead into a body. The electrical lead includes a conductive element, a stimulating electrode, a pickup electrode and a sheath. The conductive element has a proximal end and a distal end. The stimulating electrode is coupled to the distal end of the conductive element and the pickup electrode is coupled to the proximal end of the conductive element. The sheath is configured to enclose at least a portion of the conductive element. The sheath includes a reconfigurable portion that allows the length of the sheath to vary. In some embodiments, varying the length of the sheath occurs when the sheath is at least partially outside the body. In some embodiments, varying the length of the sheath occurs when the sheath is at least partially inside the body. In other embodiments, varying the length of the sheath occurs when the sheath is at least partially outside the body.

As used in this specification, the words “proximal” and “distal” refer to the direction closer to and away from, respectively, an operator (e.g., surgeon, physician, nurse, technician, etc.) who would use an electrical stimulation lead during a procedure. For example, the end of an electrical lead first to contact and/or be inserted into the patient's body would be the distal end, while the opposite end of the electrical lead (e.g., the end of the electrical lead being operated by the operator or the end of the electrical lead last to be inserted into the patient's body) would be the proximal end of the electrical lead. Therefore, the stimulating end of the lead is referred to as distal, and the pickup end of the lead is referred to as proximal.

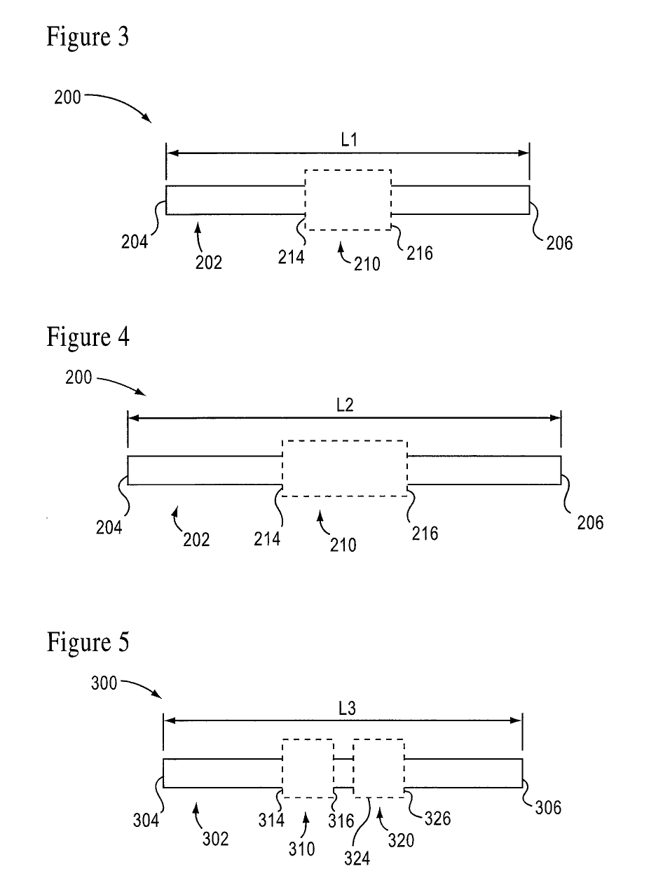

As discussed above, an electrical stimulation lead has a sheath that can be configured to move between a first configuration and a second configuration. FIGS. 3 and 4 are schematic illustrations of an electrical stimulation lead 200 with a sheath 202 in a first configuration and a second configuration, respectively. The sheath 202 is configured to at least partially enclose a conductive element (not shown in FIGS. 3 and 4) and includes a proximal portion 204, a distal portion 206 and a reconfigurable portion 210 including a proximal end portion 214 and a distal end portion 216. In the first configuration, the sheath 202 has a length L₁ defined by the distance between the proximal portion 206 and the distal portion 204. In the second configuration, the sheath 202 has a length L₂ defined in the same manner. The reconfigurable portion 210 is coupled to the sheath 202 and is configured to expand and/or contract when the sheath 202 is moved between the first configuration and the second configuration. Additionally, the proximal end portion 214 and the distal end portion 216 of the reconfigurable portion 210 move in relation to the proximal portion 204 and the distal portion 206 of the sheath 202, respectively, when the sheath 202 moves between configurations.

As shown in FIG. 4, the reconfigurable portion 210 is configured to expand when the sheath 202 is moved to the second configuration. As discussed in more detail herein, the expansion of the reconfigurable portion 210 results in the distance between the proximal end portion 214 and the distal end portion 216 increasing. Moreover, the distance between the proximal portion 204 and the distal portion 206 of the sheath 202 increases, thus resulting in a different length L₂.

In use, the length of the sheath 202 can be elongated by simply pulling or tensioning the distal portion 206 and the proximal portion 204 of the sheath 202 thus extending the reconfigurable portion 210 of the sheath 202. In some embodiments, when the reconfigurable portion 210 of the sheath 202 is extended, it can be subsequently shortened by pushing the distal portion 206 and the proximal portion 204 of the sheath 202 together thus collapsing the reconfigurable portion 210 of the sheath 202 substantially back to its original position. In some embodiments, the reconfigurable portion 210 can be partially expanded by pulling or tensioning one end of the sheath 202 as discussed in more detail herein.

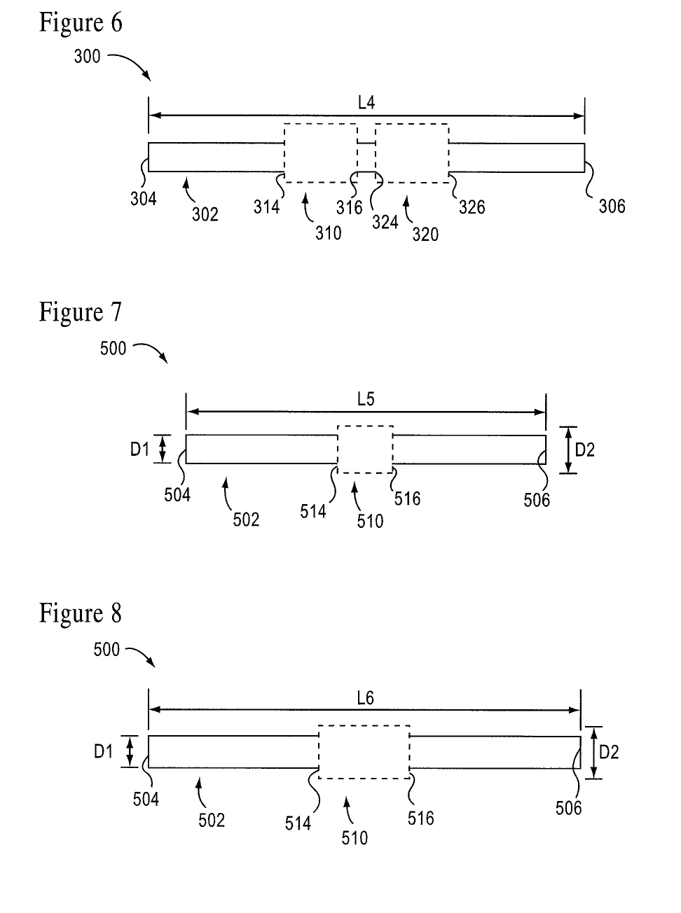

Although with respect to FIGS. 3 and 4, the sheath 202 is described and shown having only one reconfigurable portion 210, in some embodiments, a sheath can have multiple reconfigurable portions. For example, FIGS. 5 and 6 are schematic illustrations of an electrical stimulation lead 300 having a sheath 302 having a first reconfigurable portion 310 and a second reconfigurable portion 320. The sheath 302 is configured to a least partially enclose a conductive element (not shown in FIGS. 5 and 6) and includes a proximal portion 304, a distal portion 306, a first reconfigurable portion 310 and a second reconfigurable portion 320. Each of the reconfigurable portions 310, 320 have a distal end portion 316, 326 and a proximal end portion 314, 324, respectively. Reconfigurable portions 310, 320 are configured to be two separate portions of the sheath 302 and are separated from each other by some length of sheath 302. Additionally, sheath 302 is configured to move between a first configuration and a second configuration. As shown in FIGS. 5 and 6, the sheath 302 has a length L₃ when in the first configuration and a length L₄ when in the second configuration. The lengths, L₃ and L₄, are defined by the distance between the proximal portion 304 and the distal portion 306.

When the sheath 302 is in the first configuration, the first reconfigurable portion 310 and the second reconfigurable portion 320 are contracted, i.e., collapsed. When the sheath 302 moves to the second configuration, at least one of the first reconfigurable portion 310 and the second reconfigurable portion 320 expand so the sheath 302 has a length L₄. In this embodiment, length L₃ is shorter than length L₄.

FIGS. 5 and 6 illustrate both the first reconfigurable portion 310 and the second reconfigurable portion 320 expanding when the sheath 302 moves from the first configuration to the second configuration. In some embodiments, the sheath 302 can be configured so that one of the reconfigurable portions, for example, the first reconfigurable portion 310 expands while the second reconfigurable portion 320 remains collapsed when the sheath 302 moves to the second configuration. Said another way, the first reconfigurable portion 310 and the second reconfigurable portion 320 need not be expanded simultaneously. In some embodiments, the sheath 302 can move between multiple configurations.

In some embodiments, reconfigurable portion(s) can be configured to have a constant diameter when the sheath is moved from the first configuration to the second configuration. For example, FIGS. 7 and 8 illustrate an electrical stimulation lead 500 having a sheath 502 having a single reconfigurable portion 510 with a diameter D₂. The sheath 502 is configured to at least partially enclose a conductive element (not shown in FIGS. 7 and 8) and includes a proximal portion 504, a distal portion 506 and the reconfigurable portion 510. The reconfigurable portion 510 includes a proximal end portion 514 and a distal end portion 516. In a first configuration, as shown in FIG. 7, the sheath 502 has a diameter D₁ and the reconfigurable portion 510 has a diameter D₂. In the second configuration, shown in FIG. 8, the diameter D₁ of sheath 502 and the diameter D₂ of the reconfigurable portion 510 remain constant, even though the length L₆ in the second configuration is greater than the length L₅ in the first configuration.

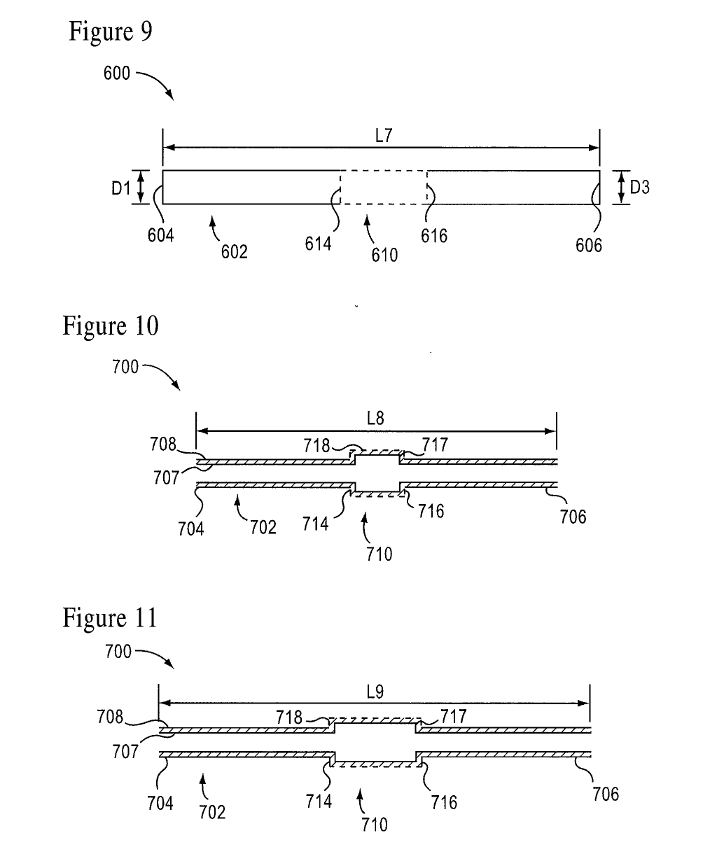

In some embodiments, the diameter D₂ of the reconfigurable portion 510 changes when the sheath 502 is moved from a first configuration to a second configuration. For example, FIG. 9 is a schematic illustration of an alternative second configuration for sheath 502, where an electrical stimulation lead 600 having a sheath 602 has a reconfigurable portion 610 with a diameter D₃. Sheath 602 is configured to at least partially enclose a conductive element (not shown in FIG. 9) and includes a proximal portion 604, a distal portion 606 and the reconfigurable portion 610. The reconfigurable portion 610 includes a proximal end portion 614 and a distal end portion 616.

In FIG. 9, the sheath 602 is in the alternative second configuration and has a length L₅ and a diameter D₁ while the reconfigurable portion 610 has a diameter D₃. In this embodiment, the diameter D₃ of the reconfigurable portion 610 is equal to the diameter D₁ of the sheath 602. However, in some embodiments, the diameter D₃ of the reconfigurable portion 610 can be greater than the diameter D₁ of the sheath 610. Additionally, in other embodiments, the diameter D₃ of the reconfigurable portion 610 can be less than the diameter D₁ of the sheath 602 when the sheath 602 is in the second configuration.

In some embodiments, a reconfigurable portion of a sheath can be configured to have a wall thickness that remains constant when the sheath is moved from a first configuration to a second configuration. For example, FIGS. 10 and 11 are schematic illustrations of an electrical stimulation lead 700 having a sheath 702 having a wall thickness including a reconfigurable portion 710 having a constant wall thickness. The sheath 702 is configured to at least partially enclose a conductive element (not shown in FIGS. 10 and 11) and includes proximal portion 704, a distal portion 706, an inner wall 707, an outer wall 708 and the reconfigurable portion 710. The reconfigurable portion includes a proximal end portion 714, a distal end portion 716, an inner wall 717 and an outer wall 718. The wall thickness of the sheath 702 is defined by the distance between the inner wall 707 and the outer wall 708. Similarly, the wall thickness of the reconfigurable portion 710 is defined by the inner wall 717 and the outer wall 718.

In the first configuration, as shown in FIG. 10, the sheath has a length L₈ and has substantially the same wall thickness as the reconfigurable portion 710. When the sheath 702 moves to the second configuration, as shown in FIG. 11, the sheath 702 has a different length L₉, but the wall thickness of the sheath 702 and the reconfigurable portion 710 remains substantially constant. In some embodiments, however, the wall thickness of the reconfigurable portion 710 changes when the sheath 702 moves from the first configuration to the second configuration.

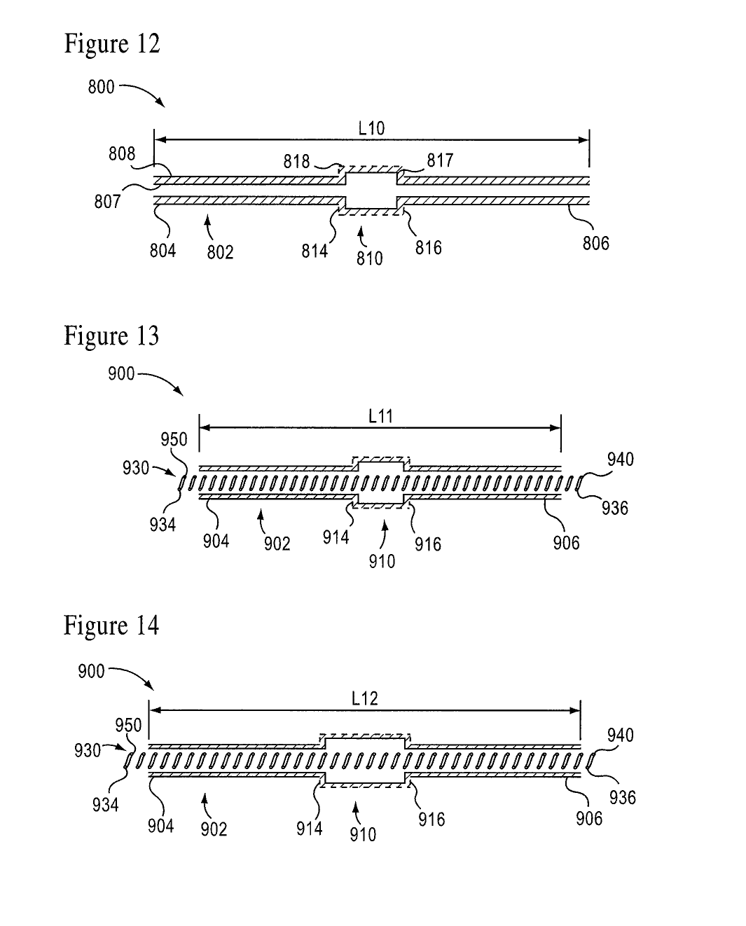

For example, FIG. 12 is a schematic illustration of the electrical stimulation lead 700 in an alternative second configuration, as represented by the electrical stimulation lead having a sheath 802. In the second configuration, the wall thickness of reconfigurable portion 810 is substantially less than the wall thickness of the reconfigurable portion 810 in the first configuration (see FIG. 10). Moreover, in some embodiments, the wall thickness of the reconfigurable portion 810 in the second configuration can be substantially greater than the wall thickness of the reconfigurable portion 810 in the first configuration.

In all of the previous embodiments, the sheaths 202, 302, 502, 602, 702 and 802 can be configured to enclose at least a portion of a conductive element. FIGS. 13 and 14 are schematic illustrations of an electrical stimulation lead 900 having a conductive element 930 at least partially enclosed within a sheath 902 in a first configuration and a second configuration, respectively. The sheath 902 includes a proximal portion 904, a distal portion 906 and a reconfigurable portion 910 including a proximal end portion 914 and a distal end portion 916. The conductive element 930 includes a proximal end 934, a distal end 936, a stimulating electrode 940 and a pickup electrode 950. The stimulating electrode 940 is coupled to the distal end 936 of the conductive element 930 and the pickup electrode 950 is coupled to the proximal end 934 of the conductive element 930.

In the first configuration, the sheath 902 has a length L₁₁ and the conductive element 930 has a length defined by the distance between its proximal end 934 and its distal end 936. In the second configuration, the reconfigurable portion 910 is expanded and the length of the sheath 902 increases to length L₁₂. Additionally, the length of the conductive element 930 also increases. Said another way, the distance between the stimulating electrode 940 and the proximal end of the conductive element 930 increases when the sheath 902 moves to the second configuration. However, in some embodiments, the length of the conductive element 930 can remain constant when the sheath 902 moves from the first configuration to the second configuration. When the length of the conductive element 930 remains constant, the distance between the stimulating electrode 940 and the proximal end 934 of the conductive element 930 also remains constant.

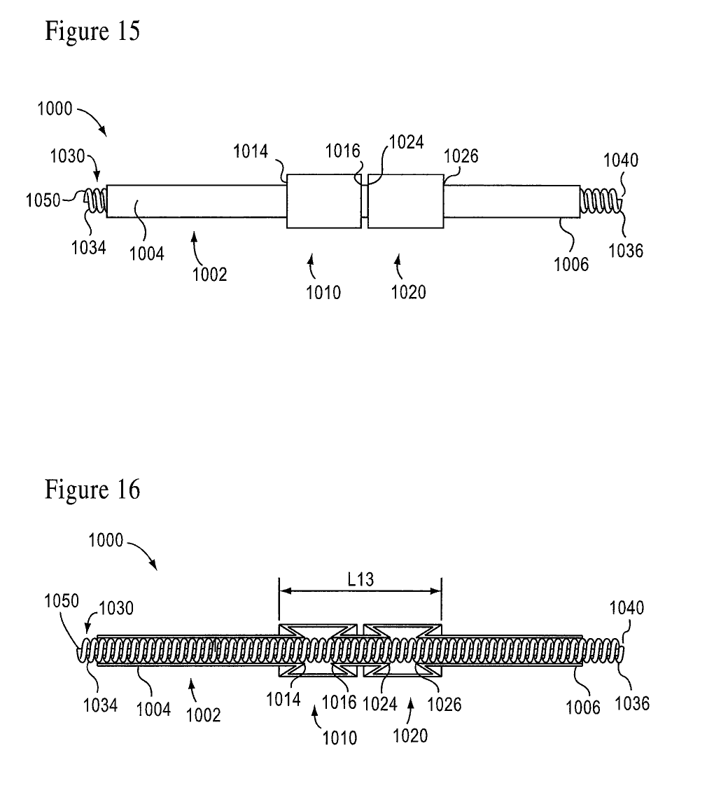

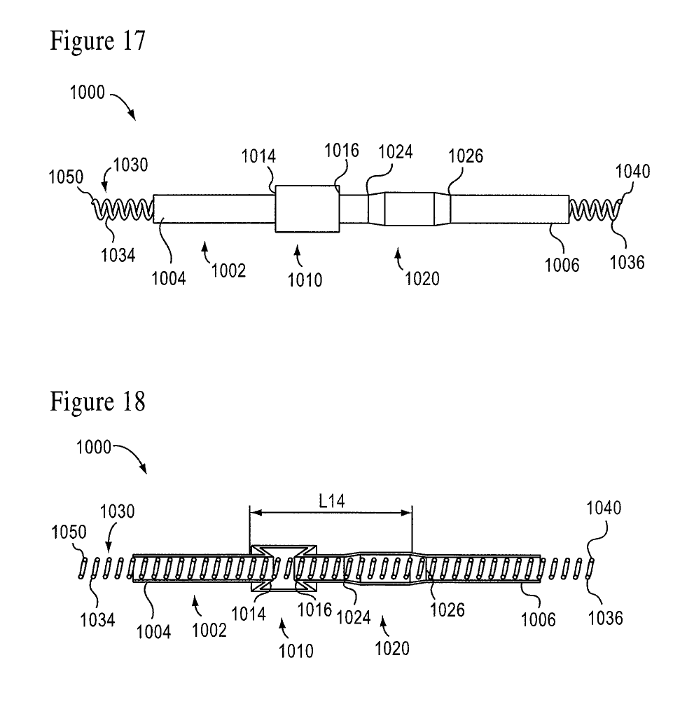

FIGS. 15 and 17 are illustrations of an electrical stimulation lead 1000 in a first configuration and a second configuration, respectively. FIGS. 16 and 18 are the cross-sectional views of the lead 1000 in the first configuration and the second configuration, respectively. The lead 1000 includes a conductive element 1030 and a sheath 1002. The sheath 1002 is configured to at least partially enclose the conductive element 1030. The sheath includes a proximal portion 1004, a distal portion 1006, a first reconfigurable portion 1010 and a second reconfigurable portion 1020. Each of the reconfigurable portions include a proximal end portion 1014, 1024 and a distal end portion 1016, 1026, respectively. The reconfigurable portions 1010, 1020 are separated from each other by some length of sheath 1002. The conductive element includes a proximal end 1034, a distal end 1036, a stimulating electrode 1040 coupled to the distal end 1036 of the conductive element 1030 and a pickup electrode 1050 coupled to the proximal end 1034 of the conductive element 1030.

In FIGS. 15 and 16, the sheath 1002 is in a first configuration where the first reconfigurable portion 1010 and the second reconfigurable portion 1020 are contracted, i.e., collapsed. FIG. 16 is a cross-sectional view of FIG. 15, illustrating the arrangement of the collapsed reconfigurable portions 1010 and 1020. While FIGS. 15-18 show the reconfigurable portions 1010 and 1020 as having a folded arrangement, in some embodiments, the reconfigurable portions 1010 and 1020 can have any number of bends, alternate furrows and/or the like, as discussed in more detail herein.

The folded arrangement of the reconfigurable portions 1010 and 1020, as shown in FIG. 16, is structured so that the proximal end portion 1014 and the distal end portion 1016 of the first reconfigurable portion 1010 are inverted within a portion of the first reconfigurable portion 1010. Likewise, the proximal end portion 1024 and the distal end portion 1026 of the second reconfigurable portion 1020 are inverted within a portion of the second reconfigurable portion 1020. As a result, the reconfigurable portions 1010 and 1020 are at least partially folded along the sheath 1002. In some embodiments, however, the folded arrangement can be reversed so that the reconfigurable portions 1010 and 1020 are folded in the opposite direction.

In FIG. 17, the sheath 1002 moves to the second configuration by expanding the second reconfigurable portion 1020. The expansion occurs when the second reconfigurable portion 1020 is unfolded. Although, FIGS. 17 and 18 show the first reconfigurable portion 1010 as remaining folded when the sheath 1002 moves to the second configuration, in some embodiments, the sheath 1002 can move to the second configuration when the first reconfigurable portion 1010 is unfolded, i.e., expanded, while the second reconfigurable portion 1020 remains folded. Said another way, the reconfigurable portions 1010 and 1020 need not be expanded simultaneously.

In the second configuration, as shown in FIGS. 17 and 18, the second reconfigurable portion 1020 is fully expanded. In some embodiments, however, the sheath 1002 can move to the second configuration when the second reconfigurable portion 1020 is only partially expanded. For example, the proximal end portion 1024 of the second reconfigurable portion 1020 can remain folded while the distal end portion 1026 of the second reconfigurable portion 1020 is extended.

When the sheath 1002 is moved between the first configuration and the second configuration, the length of the reconfigurable portions 1010 and 1020 changes. For example, when the sheath 1002 is in the first configuration, as shown in FIG. 16, the overall length L₁₃ of the reconfigurable portions 1010 and 1020 is defined by the distance between the proximal end portion 1014 of the first reconfigurable portion 1010 and the distal end portion 1026 of the second reconfigurable portion 1020. When the sheath 1002 moves to the second configuration, as shown in FIG. 18, the reconfigurable portions 1010 and 1020 have a different overall length L₁₄.

The extended length of the sheath 1002 results in an increased distance between the stimulating electrode 1040 and the proximal portion 1004 of the sheath 1002. In other words, the conductive element 1030 is elongated when the sheath 1002 moves to the second configuration.

In some embodiments, the proximal end portion 1014 of the first reconfigurable portion 1010 and the distal end portion 1026 of the second reconfigurable portion 1020 move in relation to the proximal portion 1004 and the distal portion 1006 of the sheath 1002, respectively. In such an embodiment, the distal end portion 1016 of the first reconfigurable portion and the proximal end portion 1024 of the second reconfigurable portion 1020 remain stationary.

Although FIGS. 15-18 illustrate the reconfigurable portions 1010, 1020 as having a central location along the length of the sheath 1102 and having similar lengths, in some embodiments, the first reconfigurable portion 1010 and the second reconfigurable portion 1020 can have different lengths and/or locations along the length of the sheath 1002. For example, the first reconfigurable portion 1010 can have a longer length than the second reconfigurable portion 1020. In some embodiments, the first reconfigurable portion 1010 can have a different structure than the second reconfigurable portion 1020. For example, the first reconfigurable portion 1010 can be configured in a folded arrangement and the second reconfigurable portion 1020 configured in a pleated arrangement. Moreover, in some embodiments, the first reconfigurable portion 1010 can be located near the proximal end 1004 and the second reconfigurable portion 1020 located near the distal end 1006 of the sheath 1002. The above described embodiments can be varied, allowing a large degree of flexibility and applications.

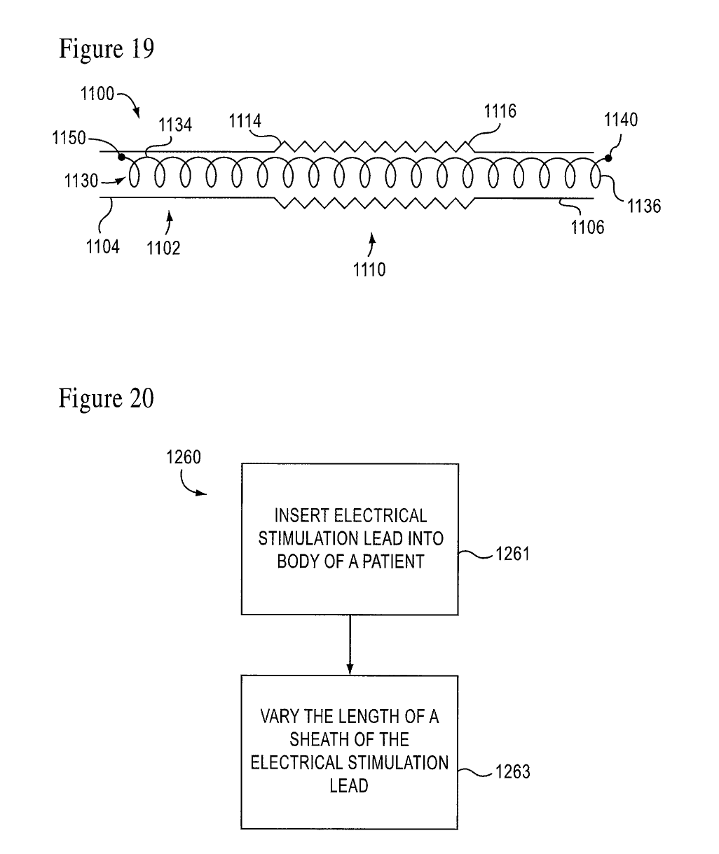

Although the reconfigurable portions 1010 and 1020 were depicted as having folded arrangements, in some embodiments, the reconfigurable portion(s) can have a corrugated arrangement. For example, in FIG. 19, an electrical stimulation lead 1100 having a sheath 1102 includes a reconfigurable portion 1110 having a corrugated arrangement. The sheath 1102 is configured to at least partially enclose a conductive element 1130 and includes a proximal portion 1104, a distal portion 1106 and the reconfigurable portion 1110. The reconfigurable portion 1110 includes a proximal end portion 1114 and a distal end portion 1116. The conductive element 1130 includes a proximal end 1134, a distal end 1136 and a stimulating electrode 1140 coupled to the distal end 1136 of the conductive element 1130 and a pickup electrode 1150 coupled to the proximal end 1134 of the conductive element 1130. In some embodiments, the entire length of the sheath 1102 can be corrugated in the same manner as reconfigurable portion 1110. In other words, the length of the sheath 1102 is substantially infinitely expandable.

The reconfigurable portion 1110 is configured to change the length of the sheath 1102. In some embodiments, for example, the reconfigurable portion 1110 consists of any number of folds, bends, alternate furrows, ridges, wrinkles, corrugations and/or the like. In this manner, when the sheath 1102 is in a first configuration the reconfigurable portion 1110 is contracted and the sheath 1102 is shortened. Said another way, the material of the reconfigurable portion 1110 is in any one of the described configurations that condense the material of the sheath 1102. When the sheath 1102 moves to a second configuration (not shown) the reconfigurable portion 1110 is expanded and the sheath 1102 is lengthened.

In some embodiments, the thickness of the material of the reconfigurable portion 1110 may vary along the length of the reconfigurable portion 1110. For example, the thickness of the material of the reconfigurable portion 1110 may be thinner at the bends or folds of the reconfigurable portion 1110.

In some embodiments, the sheath 1102 can be made of an insulative material. For example, the sheath 1102 can be made of Teflon™ FEP (DuPont). In other embodiments, the material of the sheath 1102 can be constructed to have an elastic quality, allowing the sheath 1102 to stretch and bend without significant structural deformation.

In some embodiments, the sheath 1102 can be constructed to have portions that can be torn off to adjust the length. For example, in some embodiments, the sheath can have perforated sections or the like to enable a portion of the sheath to be easily removed.

The reconfigurable portion 1110 can be a physically distinct portion coupled to sheath 1102, but in some embodiments, the sheath 1102 and the reconfigurable portion 1110 can be a monolithic structure. In other embodiments, the sheath 1102 and the reconfigurable portion 1110 can be made of the same material and coupled together. In yet other embodiments, the sheath 1102 and the reconfigurable portion 1110 can be made of at least two different materials and coupled together.

The reconfigurable portions can undergo plastic or elastic deformation with moving to the second configuration. In some embodiments, the sheaths 202, 302, 502, 602, 702, 802, 902, 1002 and 1102 can move from the first configuration to the second configuration and back, again, to the first configuration. In other embodiments, the sheaths 202, 302, 502, 602, 702, 802, 902, 1002 and 1102 can only move from the first configuration to the second configuration. In this manner, the length of the sheaths 202, 302, 502, 602, 702, 802, 902, 1002 and 1102 is irreversible once the sheaths 202, 302, 502, 602, 702, 802, 902, 1002 and 1102 are in the second configuration.

Although embodiments described above have a sheath configured to move between a first configuration and a second configuration, in some embodiments, the sheath can be configured to move between multiple configurations. For example, the sheath can be configured to move between three configurations. In the first configuration, the reconfigurable portion can be condensed. In the second configuration, the reconfigurable portion can be partially expanded and in the third configuration, the reconfigurable portion can be fully expanded. In another example, a sheath, having two reconfigurable portions, can have a first configuration were both reconfigurable portions are condensed, a second configuration where only one of the reconfigurable portions are expanded and a third configuration where the second reconfigurable portion is expanded.

The reconfigurable portion can be configured to have any length and to be positioned at any location along the length of the sheath. Although, for example, FIG. 19 illustrates the reconfigurable portion 1110 positioned in a central location on the sheath 1102, in some embodiments, for example, the reconfigurable portion 1110 can be positioned closer to the proximal end 1104 of the sheath 1102. Additionally, the reconfigurable portion 1110 has a length defined by the distance between the proximal end portion 1114 and the distal end portion 1116 of the reconfigurable portion 1110. In some embodiments, the length of the reconfigurable portion 1110 can be longer than in others.

In some embodiments, as shown in FIG. 20, a method 1260 of inserting an electrical stimulation lead within a body of a patient is described. The electrical stimulation lead has a sheath, a conductive element, a stimulation electrode and a pickup electrode. The sheath is configured to enclose at least a portion of the conductive element and includes a reconfigurable portion. The method 1260 includes inserting 1261 the electrical stimulation lead into the body of a patient. The method includes varying 1263 the length of the sheath by moving the reconfigurable portion between a first configuration and a second reconfiguration so that the length of the sheath in the first configuration is different from the length of the sheath in the second configuration.

In some embodiments, the length of the sheath can be varied 1263 before the electrical stimulation lead is inserted 1261 into the body of the patient. In other embodiments, the length of the sheath can be varied 1263 when the sheath is at least partially inside the body. In yet other embodiments, the length of the sheath can be varied 1263 when the sheath is at least partially outside the body.

Although the sheath is illustrated as surrounding the conductive element, the conductive element can be embedded in the sheath.

Although the sheaths 202, 302, 502, 602, 702, 802, 902, 1002 and 1102 are described as moving between a first configuration and a second configuration, it should be understood that each of the discussed reconfigurable portions are moveable between the first configuration and the second configuration. Accordingly, the disclosed electrical stimulation leads are moveable between the first configuration and the second configuration.

Although the electrical stimulation leads are shown and described herein include one or two reconfigurable portions, each of the electrical stimulation leads can have any number of reconfigurable portions. Moreover, any of the reconfigurable portions can be used in any combination with any electrical stimulation lead.

Although the conductive element is illustrated as having a pickup electrode coupled to the proximal end of the conductive element, the proximal end of the conductive element can include a connector.

While various embodiments of the invention have been described above, it should be understood that they have been presented by way of example only, and not limitation. Thus, the breadth and scope of the invention should not be limited by any of the above-described embodiments, but should be defined only in accordance with the claims and their equivalents.

What is claimed is:

1. An apparatus, comprising:
a conductive element having a proximal end and a distal end, the conductive element having a length defined between the proximal end and the distal end;
a stimulating electrode coupled to the distal end of the conductive element; and
a sheath configured to enclose at least a portion of the conductive element, the sheath having a reconfigurable portion moveable between a first configuration and a second configuration, the sheath having a first length when the reconfigurable portion is in the first configuration and a second length when the reconfigurable portion is in the second configuration.

2. The apparatus of claim 1, wherein the sheath is a monolithic structure.

3. The apparatus of claim 1, wherein the length of the conductive element is substantially constant when the reconfigurable portion of the sheath is moved between the first configuration and the second configuration.

4. The apparatus of claim 1, wherein the conductive element has a first length when the reconfigurable portion of the sheath is in the first configuration and a second length when the reconfigurable portion of the sheath is in the second configuration.

5. The apparatus of claim 1, wherein the reconfigurable portion of the sheath has multiple reconfigurable portions.

6. The apparatus of claim 1, wherein the sheath includes multiple reconfigurable portions spaced apart from each other.

7. The apparatus of claim 1, wherein the reconfigurable portion of the sheath is configured to be selectively variable through a range of lengths.

8. The apparatus of claim 1, wherein the reconfigurable portion of the sheath has a constant diameter when the reconfigurable portion moves between the first configuration and the second configuration.

9. The apparatus of claim 1, wherein the reconfigurable portion of the sheath has a first diameter when the reconfigurable portion is in the first configuration and a second diameter when the reconfigurable portion is in the second configuration.

10. The apparatus of claim 1, wherein the sheath has a constant wall thickness when the reconfigurable portion moves between the first configuration and the second configuration.

11. The apparatus of claim 1, wherein the sheath has a first wall thickness when the reconfigurable portion is in the first configuration and a second wall thickness with the reconfigurable portion is in the second configuration.

12. The apparatus of claim 1, wherein the stimulating electrode is a first distance from a proximal end of the sheath when the reconfigurable portion is in the first configuration and a second distance from the proximal end of the sheath when the reconfigurable portion is in the second configuration.

13. The apparatus of claim 1, further comprising a pickup electrode coupled to the proximal end of the conductive element.

14. An apparatus comprising:
a conductive element having a proximal end and a distal end, the conductive element having a length defined between the proximal end and the distal end;
a stimulating electrode coupled to the distal end of the conductive element; and
a monolithic sheath configured to enclose at least a portion of the conductive element, at least a portion of the sheath being reconfigurable to vary a length of the sheath.

15. The apparatus of claim 14, wherein the at least a portion of the sheath is reconfigurable from a first configuration to a second configuration, the length of the sheath in the second configuration is longer than the length of the sheath in the first configuration.

16. The apparatus of claim 14, wherein the at least a portion of the sheath is reconfigurable from a first configuration to a second configuration, the length of the sheath in the first configuration is longer than the length of the sheath in the second configuration.

17. The apparatus of claim 14, wherein the length of the sheath is reconfigurable to be shortened by removing at least a portion of the sheath.

18. The apparatus of claim 14, wherein the at least a portion of the sheath being reconfigurable includes multiple reconfigurable portions.

19. The apparatus of claim 14, wherein the at least a portion of the sheath being reconfigurable includes multiple reconfigurable portions spaced apart from each other.

20. The apparatus of claim 14, wherein the length of the conductive element is substantially constant when the at least a portion of the sheath is reconfigurable between a first configuration and a second configuration.

21. The apparatus of claim 14, wherein the conductive element has a first length when the at least a portion of the sheath is reconfigurable in a first configuration and a second length when the at least a portion of the sheath is reconfigurable in a second configuration.

22. The apparatus of claim 14, wherein the at least a portion of the sheath being reconfigurable has a constant diameter when the at least a portion of the sheath is reconfigurable between a first configuration and a second configuration.

23. The apparatus of claim 14, wherein the at least a portion of the sheath being reconfigurable has a first diameter when the at least a portion of the sheath is reconfigurable in a first configuration and a second diameter when the at least a portion of the sheath is reconfigurable in a second configuration.

24. The apparatus of claim 14, wherein the sheath has a constant wall thickness when the at least a portion of the sheath is reconfigurable between a first configuration and a second configuration.

25. The apparatus of claim 14, wherein the sheath has a first wall thickness when the at least a portion of the sheath being reconfigurable is in a first configuration and a second wall thickness with the at least a portion of the sheath being reconfigurable is in a second configuration.

26. The apparatus of claim 14, wherein the stimulating electrode is a first distance from a proximal end of the sheath when the at least a portion of the sheath is reconfigurable in a first configuration and a second distance from the proximal end of the sheath when the at least a portion of the sheath is reconfigurable in a second configuration.

27. The apparatus of claim 14, further comprising a pickup electrode coupled to the proximal end of the conductive element.

28. A method, comprising:
inserting an electrical stimulation lead into a body, the electrical stimulation lead having a conductive element, a stimulating electrode and a sheath configured to enclose at least a portion of the conductive element; and
varying a length of the sheath by moving a reconfigurable portion of the sheath between a first configuration and a second configuration, the length of the sheath in the first configuration being different from the length of the sheath in the second configuration.

29. The method of claim 28, wherein the varying the length of the sheath occurs when the sheath is at least partially outside the body.

30. The method of claim 28, wherein the varying the length of the sheath occurs when the sheath is at least partially inside the body.

31. The method of claim 28, wherein the length of the sheath in the second configuration is longer than the length of the sheath in the first configuration.

32. The method of claim 28, wherein the length of the sheath in the first configuration is longer than the length of the sheath in the second configuration.

33. The method of claim 28, wherein the electrical stimulation lead includes a pickup electrode.

34. A kit, comprising:
an electrical stimulator lead, the electrical stimulator lead having a conductive element, a stimulating electrode and a sheath configured to enclose at least a portion of the conductive element, the sheath having a reconfigurable portion moveable between a first configuration and a second configuration, the sheath having a first length when the reconfigurable portion is in the first configuration and a second length when the reconfigurable portion is in the second configuration.

35. The kit of claim 34, wherein the length of the sheath in the second configuration is longer than the length of the sheath in the first configuration.

36. The kit of claim 34, wherein the length of the sheath in the first configuration is longer than the length of the sheath in the second configuration.

37. The kit of claim 34, wherein the reconfigurable portion of the sheath is configured to be selectively variable through a range of lengths.

38. The kit of claim 34, wherein the sheath is a monolithic structure.

39. The kit of claim 34, wherein the electrical stimulator lead includes a pickup electrode.
